(19) United States
(12) Patent Application Publication
(10) Pub. No.: US 2025/0380947 A1
(43) Pub. Date: Dec. 18, 2025

(54) METHODS FOR MAKING MEDICAL DEVICES THAT INCLUDE A SHAPE MEMORY FOAM COMPONENT

(71) Applicant: BOSTON SCIENTIFIC SCIMED, INC., Maple Grove, MN (US)

(72) Inventors: Scott Andrew McCullough, Plymouth, MN (US); Patrick Willoughby, Shoreview, MN (US)

(73) Assignee: BOSTON SCIENTIFIC SCIMED, INC., MAPLE GROVE, MN (US)

(21) Appl. No.: 19/238,288

(22) Filed: Jun. 13, 2025

Related U.S. Application Data
(60) Provisional application No. 63/660,348, filed on Jun. 14, 2024.

(51) Int. Cl. A61B 17/12

(52) U.S. Cl. CPC A61B 17/12122; A61B 17/12181

(57) ABSTRACT

A medical device may include a first component and a shape memory foam component that is secured relative to the first component. The medical device may be formed by forming a foamable solution that over time progresses from an initial liquid pre-foam state to a subsequent mid-foam state in which the foamable solution is partially reacted and to a final foam state in which the foamable solution is fully reacted and then applying the foamable solution to the first component during one of the liquid pre-foam state, the subsequent mid-foam state and the final foam state.

### CROSS-REFERENCE TO RELATED APPLICATION

This application is a continuation of U.S. Patent Application Ser. No. 63/660,348, filed Jun. 14, 2024, entitled “METHODS FOR MAKING MEDICAL DEVICES THAT INCLUDE A SHAPE MEMORY FORM COMPONENT”, which is incorporated by reference herein in its entirety.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a method of making a medical device that includes a first component and a shape memory foam component secured relative to the first component. The method includes forming a foamable solution that over time progresses from an initial liquid pre-foam state to a subsequent mid-foam state in which the foamable solution is partially reacted and to a final foam state in which the foamable solution is fully reacted. The foamable solution is applied to the first component during one of the liquid pre-foam state, the subsequent mid-foam state and the final foam state.

Alternatively or additionally, the method may further include allowing the foamable solution to progress to the final foam state.

Alternatively or additionally, the pre-foam state may correspond to the foamable solution being less than about thirty percent reacted.

Alternatively or additionally, the mid-foam state may correspond to the foamable solution being at least about thirty percent reacted and no more than about seventy percent reacted.

Alternatively or additionally, the final foam state may correspond to the foamable solution being more than about seventy percent reacted.

Alternatively or additionally, the pre-foam state may correspond to the foamable solution being less than twenty percent reacted, the mid-foam state may correspond to the foamable solution being at least twenty percent reacted and no more than about eighty percent reacted, and the final foam state may correspond to the foamable solution being more than about eighty percent reacted.

Alternatively or additionally, the method may further include applying a binding solution to the first component prior to applying the foam able solution to the first component.

Alternatively or additionally, the binding solution may include the same monometers as the foamable solution but without any blowing agents.

Alternatively or additionally, the foamable solution may include polyol or isocyanate monomers.

Alternatively or additionally, the first component may include an occlusive covering for a left atrial appendage closure (LAAC) device.

Alternatively or additionally, applying the foamable solution may include applying the foamable solution to a face of the occlusive covering during the foamable solution's initial pre-foam state.

Alternatively or additionally, applying the foamable solution may include applying the foamable solution to a periphery of the occlusive covering during the foamable solution's mid-foam state.

Alternatively or additionally, the first component may include a first block of foam.

Alternatively or additionally, the first component may include an anchoring system.

Alternatively or additionally, the first component may include a polymeric component and the method may further include etching the polymeric component before applying the foamable solution.

Alternatively or additionally, the foamable solution may create a shape memory foam.

Another example may be found in a medical device that may be produced by forming a foamable solution that over time progresses from an initial liquid pre-foam state to a subsequent mid-foam state in which the foamable solution is partially reacted and to a final foam state in which the foamable solution is fully reacted. The foamable solution may be applied to a first component during one of the liquid pre-foam state, the subsequent mid-foam state and the final foam state in order to form the medical device.

Another example may be found in a method of adding a shape memory polymer foam component to an implantable medical device. The method includes forming a foamable solution, allowing the foamable solution to reach one of an initial liquid pre-foam state, a subsequent mid-foam state and a final foam state, and contacting the implantable medical device with the foamable solution once the foamable solution has reached the desired state.

Alternatively or additionally, the implantable medical device may include a left atrial appendage closure (LAAC) device.

Another example may be found in a method of adding a shape memory foam component to a left atrial appendage closure (LAAC) device. The method includes forming a foamable solution that over time progresses from an initial liquid pre-foam state to a subsequent mid-foam state in which the foamable solution is partially reacted and to a final foam state in which the foamable solution is fully reacted, and applying the foamable solution to the LAAC device during one of the liquid pre-foam state, the subsequent mid-foam state and the final foam state.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative LAAC device;

FIG. 2 is a schematic side view of the illustrative occlusive covering forming part of the illustrative LAAC device of FIG. 1, with a foamable solution applied to a front face of the occlusive covering;

FIG. 3 is a schematic side view of the occlusive covering of FIG. 2, with the foamable solution shown fully reacted;

FIG. 4 is a schematic side view of the occlusive covering forming part of the illustrative LAAC device of FIG. 1, with a foamable solution applied around a periphery of the occlusive covering;

FIG. 5 is a cross-sectional view taken along the line 5-5 of FIG. 4;

FIG. 6 is a schematic side view of the occlusive covering forming part of the illustrative LAAC device of FIG. 1, with the foamable solution shown fully reacted;

FIG. 7 is a cross-sectional view taken along the line 7-7 of FIG. 6;

FIG. 8 is a schematic side view of an illustrative medical device including a first component and a foamable solution applied to the first component; and

FIG. 9 is a schematic side view of the illustrative medical device of FIG. 8, with the foamable solution shown fully reacted.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Various types of foam, including but not limited to shape memory foam, may be used in a variety of different medical devices. Medical devices may be made from one or more pieces of foam that are secured together in order to create a more complex geometry, for example. Foam may be added to other components. Foam may be added to one or more surfaces of another component, such as metal surfaces, polymeric surfaces or even other foam surfaces. In some cases, foam such as a shape memory foam may be used as a connecting element between other components of a device. Foam may be used for positioning one device component relative to another device component. Foam may be used to allow compression between components and/or to increase stiffness between components.

In some cases, foam components, including shape memory foam components, may include an anchoring system. The anchoring system may be metallic or polymeric. The anchoring system may include shape memory foam in a solid form, such as a polymer that cures without using any blowing agents. As an example, a shape memory foam may be formed around and about a metallic or polymeric anchoring system. After the shape memory foam cures and envelops the anchoring system, an abrasive or cutting process may be used to remove portions of the shape memory foam that occlude the anchors, hooks or barbs forming part of the anchoring system. As a result, the anchors, hooks or barbs are exposed and are able to anchor the shape memory foam device within a desired anatomy.

In some cases, foams including shape memory foams may be used to provide sealing surfaces such as proximal sealing surfaces. Foams including shape memory foams may be used to provide textured sealing features. Foams including shape memory foams may be used to attach structures to a foam implant that aid in deployment of the device. Structures may be applied to a foam implant in order to avoid premature expansion of the shape memory foam. In some cases, components may be adhered together using an adhesive between the two or more components. In some cases, additive manufacturing methods such as 3D printing may be used to create a foam structure on another structure. In some cases, the mixing and/or the extrusion time may be controlled in order to control whether the foam solution used in the 3D printing is applied before the foam solution begins to foam, or is applied during the middle of the foaming process. The foam structure may self-adhere as it is being 3D printed. In some cases, a separate adhesive component may be applied as part of the 3D printing process.

In some cases, a polymeric material may be used to attach additional components and/or features to a shape memory foam substrate. In some cases, the binding material may be the same composition as the shape memory foam substrate. As an example, a foamable solution may be applied to the shape memory foam substrate and another component to be attached to the shape memory foam substrate. The foamable solution may be allowed to foam while the shape memory foam substrate and the other component remain in contact with each other. As will be discussed, a foamable solution is a solution that includes the monomers, catalysts and blowing agents that will react to form a shape memory polymer foam. As will be discussed, a foamable solution may also include other materials such as surfactants. In some cases, a foamable solution may be applied to one or both components and the foamable solution may be allowed to begin foaming. Partway through the foaming process, the components may be brought into contact with each other and held in contact with each other while the foaming process continues. In some cases, this may bind the components together.

In some cases, several components may be bound together using a binding material. In some cases, a foamable solution may include a binding material that can then be used to bind directly to another component or to bind the foamable solution to a component. In some cases, binding material may include the same monomers as were used in creating the shape memory foam substrate but may be reacted without foaming to yield a solid polymer. In some cases, this may mean reacting the monomers in the presence of one or more catalysts and in the absence of any chemical or physical blowing agents. A polymeric film may be produced using the same monomers without foaming. The solution may still include surfactant(s) and/or catalyst(s), even without blowing agents. As an example, a polyurethane solution may be prepared without blowing agents and may be applied to the components to be attached together. The polyurethane solution may be allowed to fully react while the components remain in contact with each other. In some cases, a substrate may be primed with a prepolymer solution and subsequently dipped into an aqueous solution in order to create a polyurethane-urea coating.

In some cases, a binding material may be an elastomeric polymer. The elastomeric polymer may be a thermoset material that is cured in place. The elastomeric polymer may be a thermoplastic material that is dissolved in solvent and deposited at an interface between two components. The solvent may then be evaporated off. In some cases, a shape memory foam may be used to bind two components, neither of which are shape memory polymer foams.

As an example, a foamed polyurethane may be composed of several components, including monomers, chemical blowing agents, physical blowing agents, surfactants and catalysts. A solution including these components, prior to foaming, may be considered as being a foamable solution. Chemical blowing agents undergo a chemical change to generate gas that helps to blow the foam. Chemical blowing agents may react with monomers in solution or chemically degrade to produce gas. Physical blowing agents undergo a physical change (liquid to vapor) to generate gas. In some cases, catalysts may facilitate polyurethane bond formation and some chemical reactions.

Examples of suitable monomers for making shape memory polymer foams include polyols and isocyanates. Examples of polyols that are suitable for making shape memory foam polymers include N,N,N′,N′-tetrakis(2-hydroxypropyl)ethylenediamine, triethanolamine, diethanolamine, dipropylene glycol, 5-amino-2,4,6-triiodoisophthalic acid, 3-methyl-1,5-pentanediol, gadopentetic acid, 2-butyl-2-ethyl-1,3-propanediol, and 1,2,6-hexanetriol. Examples of isocyanates that are suitable for making shape memory polymer foams include hexamethylene diisocyanate, 2,2,4- and 2,4,4-trimethylhexamethylene diisocyanate, isophorone diisocyanate, and 1,3-bis(isocyanatomethyl)cyclohexane.

Examples of suitable chemical blowing agents include, but are not limited to, water, sodium bicarbonate, and azodicarbonamide. Examples of suitable physical blowing agents include, but are not limited to, acetone, dimethoxymethane, methyl formate, ethanol, other high-vapor pressure solvents, or specialty commercial physical blowing agents. In some cases, a foamable solution may include a surfactant such as a silicone surfactant such as that commercially available from Evonik under the name TEGOSTAB® B 8418. Other surfactants are also contemplated.

Examples of suitable catalysts include tin catalysts and amine catalysts. Examples of suitable tin catalysts include 2-ethylhexyl 4,4-dibutyl-10-ethyl-7-oxo-8-oxa-3,5-dithia-4-stannatetradecanoate, and Evonik DABCO T131. Examples of suitable amine catalysts include 1,4-diazabicyclo [2,2,2] octane, 1,1,4,7,7-pentamethyldiethylenetriamine, 2,6,10-trimethyl-2,6,10-triazaundecane, bis [2-(N,N-dimethylamino)ethyl)] ether, Evonik DABCO BL 11 and Evonik DABCO BL 22.

A foamable solution may be prepared and may be used at any of several different steps during the reaction or foaming process. A foamable solution is initially a liquid. As reagents react, gas is produced and the solution begins to cure, forming a more solid porous structure. Prior to fully reacting, the foam may be tacky, and may be considered as having a “green stage” in which free reactive groups are still present. When unreacted, the foamable solution is most reactive to whatever substrate it is applied to, but it can be difficult to control. The foamable solution may be considered as going through several steps, as outlined below, and may be applied to a substrate at any of Step One, Step Two, or Step Three:
Step Zero—the individual components of the foamable solution are combined in a solution.
Step One—the foamable solution is a liquid pre-foam.
Step Two—the foamable solution is partially reacted, and has begun to form a foam.
Step Three—the foamable solution is fully reacted, and a foam has been created.

In some cases, a pre-polymer or quasi-pre-polymer may be prepared for polyurethane synthesis. For polyurethanes, a pre-polymer refers to a reaction product formed by reacting polyols and isocyanates such that no free monomeric isocyanate is present in solution. A polyurethane pre-polymer may still include reactive isocyanate functional groups that are bound to a polymeric intermediate. For polyurethanes, a quasi-pre-polymer refers to a reaction product formed by reacting polyols and isocyanates such that free monomeric isocyanate is still present in solution. For example, a solution including polyol monomers with excess isocyanate may be prepared. The solution may include reactive isocyanate functional groups available in solution. In some cases, the pre-polymer solution may be applied to a substrate, and then the substrate may be subjected to an aqueous solution to cause reaction.

Shape memory polymer foams may be adhered to a substrate using various approaches. In some cases, the viscosity of the foamable solution may be modified through the choice and quantities of monomers, surfactants, physical blowing agents, chemical blowing agents, and/or other manufacturing aids that are added. Tuning the viscosity can aid in deposition and adherence of the foamable solution to the substrate. In some cases, the shape memory polymer foams may be adhered using any of a one-component approach, a two-component approach and via chemical crosslinking.

The one-component approach includes applying un-reacted, un-foamed foamable solution to a textured or porous substrate (such as PET (polyethylene terephthalate) fabric or another piece of shape memory polymer foam). Allowing the foamable solution to foam on and around the textured/porous substrate will physically constrain elements of the substrate within the foam, thereby physically adhering the foam to the substrate.

In the two-component approach, isocyanate, pre-polymer or quasi-pre-polymer is applied to the substrate as a primer layer. A polyol solution may then be applied. The polyol solution will react with free isocyanate functional groups and create a solid shape memory polymer foam. As another example, an aqueous solution may be applied, and the water may be allowed to react with free isocyanate functional groups to yield a textured and foamed shape memory polymer foam. In another example of the two-component approach, a dry polyol solution may be applied as a primer layer to the substrate in a single step. Isocyanate or pre-polymer may be applied to the primed substrate and allowed to react to form a solid polymer coating. If the primer layer is an aqueous polyol solution, addition of free isocyanate functional groups would yield a foamed coating.

In some cases, application of isocyanate, pre-polymer or quasi-pre-polymer to the substrate may be aided by pretreating the substrate to facilitate better adhesion between the primer and substrate, potentially through modified surface chemistry (chemical cross-linking, primer, etching, etc., to yield free reactive groups), modified hydrophilicity or hydrophobicity to improve wetting, and increasing surface area.

In some cases, chemical cross-linking may be used. This may involve etching a polymeric substrate, such as esters including PET, through oxidative degradation to yield free hydroxyl groups that can chemically bond to free isocyanate groups. In some cases, a multifunctional isocyanate in the form of a pre-polymer, a quasi-pre-polymer or a pure multifunctional isocyanate may be added during synthesis of a shape memory polymer foam in order to chemically cross-link the foam to a substrate.

The addition of shape memory polymer foams to medical devices may include a variety of different use cases. As an example, a metal anchoring system may be attached to a block of shape memory polymer foam by synthesizing the foam around the anchor and then trimming away excess foam to expose the anchoring system.

Shape memory polymer foam may be applied in a particular geometry using a 3D printing system to the sizes and/or top of a LAAC device PET skirt in order to improve the possible seal between the LAAC device and the LAA in which the LAAC device will be implanted. The shape memory polymer foam may also provide a scaffold for tissue ingrowth. A solid shape memory polymer may be added to the PET skirt, either along a distal edge of the fabric or in a geometry that is similar to the frame. The shape memory polymer may be added in a compressed form for loading into a delivery sheath, but would allow for the PET fabric to maintain an “expanded” shape to aid in proper device expansion and seal improvement. Shape memory polymer foam may be added around a core-wire attachment mechanism to reduce exposed metal on the proximal surface of the LAAC device. A piece of shape memory polymer foam may be coated with a layer of a relatively stiffer and higher density shape memory polymer foam in order to create a device with a higher radial force for improved sealing. A piece of low-density non-shape memory foam may be coated with a layer of shape-memory foam to maintain shape-memory programming for case of deliverability with reduced total material mass compared to an homogenous foam device. Shape memory polymer or foam solution may be injected within specific regions of a piece of open-cell shape memory foam to create local areas of higher density for increased stiffness to provide improved structural support or radial force of the device.

A medical device may include a first component and a shape memory foam component that is secured relative to the first component. A foamable solution may be formed that, over time, progresses from an initial liquid pre-foam state to a subsequent mid-foam state in which the foamable solution is partially reacted and to a final foam state in which the foamable solution is fully reacted. The foamable solution may be applied to the first component during one of the liquid pre-foam state, the subsequent mid-foam state and the final foam state. The foamable solution may be allowed to react to completion, i.e., to the final foam state. As an example, the pre-foam state may correspond to the foamable solution being less than about twenty percent reacted, or less than about thirty percent reacted. The mid-foam state may correspond to the foamable solution being at least about twenty percent reacted or at least about 30 percent reacted and no more than about seventy percent reacted or no more than about eighty percent reacted. The final foam state may correspond to the foamable solution being more than seventy percent reacted, or more than about eighty percent reacted.

In some cases, a binding solution may be applied to the first component prior to applying the foamable solution to the first component. As an example, the binding solution may include the same monomers as the foamable solution but without any blowing agents. The foamable solution may include polyol or isocyanate monomers. The first component may include an occlusive covering for a left atrial appendage closure (LAAC) device. Applying the foamable solution may include applying the foamable solution to a face of the occlusive covering during the foamable solution's initial pre-foam state. Applying the foamable solution may include applying the foamable solution to a periphery of the occlusive covering during the foamable solution's mid-foam state. The first component may include a first block of foam. The first component may include an anchoring system. The first component may include a polymeric component and the method may further include etching the polymeric component before applying the foamable solution. The foamable solution may create a shape memory polymer foam.

A shape memory polymer foam component may be added to an implantable medical device. A foamable solution is formed, and is allowed to reach one of an initial liquid pre-foam state, a subsequent mid-foam state and a final foam state. The implantable medical device is contacted with the foamable solution once the foamable solution has reached the desired state. A variety of different implantable medical devices may benefit from inclusion of a shape memory polymer foam component, including shape memory polymer foam components formed via application of a foamable solution to the implantable medical device. A shape memory polymer foam component may change the size or shape of an implantable medical device, for example. A shape memory polymer foam component may be used to attach additional components to an implantable medical device. As an example, the implantable medical device may include a left atrial appendage closure (LAAC) device. Other exemplary implantable medical devices may include venous or vascular stents, replacement heart valves and surgical mesh devices, for example.

A shape memory polymer foam component may be added to a left atrial appendage closure (LAAC) device. A foamable solution may be formed that, over time, progresses from an initial liquid pre-foam state to a subsequent mid-foam state in which the foamable solution is partially reacted and to a final foam state in which the foamable solution is fully reacted. The foamable solution is applied to the LAAC device during one of the liquid pre-foam state, the subsequent mid-foam state and the final foam state.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly (beta-hydroxybutyrate), polygluconate including poly-D-gluconate, poly-L-gluconate, poly-D,L-gluconate, poly (epsilon-caprolactone), poly (delta-valerolactone), poly (p-dioxanone), poly (lactide-co-glycolide) (PLGA), poly (lactide-codelta-valerolactone), poly (lactide-co-epsilon-caprolactone), poly (lactide-co-beta-malic acid), poly (beta-hydroxybutyrate-co-beta hydroxyvalerate), poly [1,3bis (p-carboxyphenoxy) propane-co-sebacic acid], and poly (sebacic acid-co-fumaric acid); (b) polycarbonate homopolymers and copolymers such as poly (trimethylene carbonate), poly (lactide-co-trimethylene carbonate) and poly (glycolide-co-trimethylene carbonate); (c) poly (ortho ester homopolymers and copolymers such as those synthesized by copolymerzation of various diketene acetals and diols; (d) polyanhydride homopolymers and copolymers such as poly (adipic anhydride), poly (suberic anhydride), poly (sebacic anhydride), poly (dodecanedioic anhydride), poly (maleic anhydride), poly [1,3-bis-(p-carboxyphenoxy) methane anhydride], and poly [alpha,omega-bis (p-carboxyphenoxy)alkane anhydride] such as poly [1,3-bis (p-carboxyphenoxy) propane anhydride] and poly [1,3-bis (p-carboxyphenoxy) hexane anhydride]; (c) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly (ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

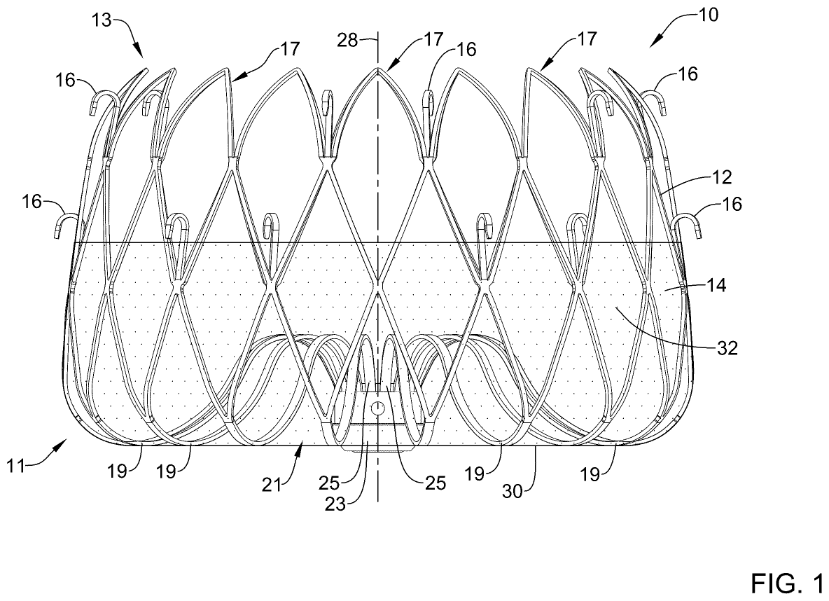

FIG. 1 is a side view of an illustrative LAAC device 10 that may be adapted to be implanted within a patient's LAA in order to occlude the patient's LAA and thus reduce or even eliminate the possibility for a blood clot within the LAA to exit the LAA and enter the patient's vasculature. An exemplary LAAC device includes WATCHMAN™ FLX available from Boston Scientific.

The LAAC device 10 may include an expandable framework 12. The expandable framework 12 may include a proximal end region 11 and a distal end region 13. FIG. 1 further illustrates that the expandable framework 12 may include one or more projections 17 extending in a proximal-to-distal direction. In some instances (such as that shown in FIG. 1), a plurality of projections 17 may extend circumferentially around a longitudinal axis 28 of the expandable framework 12. In other words, in some examples the projections 17 may resemble the peaks of a “crown” extending circumferentially around a longitudinal axis 28 of the expandable framework 12. While the above discussion (and the illustration shown in FIG. 1), shows a plurality of projections 17, it is contemplated that the LAAC device 10 may include 1, 2, 3, 4, 5, 6, 7, 8, 9, 10, 11, 12 or more individual projections 17 disposed in a variety of arrangements along the expandable framework 12.

Additionally, FIG. 1 illustrates that the proximal end region 11 of the expandable framework 12 may include a plurality of support members 19 extending circumferentially around the longitudinal axis 28 of the expandable framework 12. FIG. 1 illustrates that that plurality of support members 19 may include one or more curved portions which are shaped such that they define a “recess” 21 extending distally into the expandable framework 12. As illustrated in FIG. 1, the recess 21 may extend circumferentially around the longitudinal axis 28. Further, FIG. 1 illustrates that each of the plurality of support members 19 may include a first end 25 which is attached to a central hub 23. It can be appreciated that the central hub 23 may be aligned along the longitudinal axis 28 of the expandable framework 12. FIG. 1 illustrates that the hub 23 may be positioned such that it lies within the recess 21 defined by the plurality of support members 19.

The LAAC device 10 may also include an occlusive member 14 disposed on, disposed over, disposed about, or covering at least a portion of the expandable framework 12. In some instances, the occlusive member 14 may be disposed on, disposed over, disposed about or cover at least a portion of an outer (or outwardly-facing) surface of the expandable framework 12. FIG. 1 further illustrates that the occlusive member 14 may extend only partially along the longitudinal extent of the expandable framework 12. However, this is not intended to be limiting. Rather, the occlusive member 14 may extend along the longitudinal extent of the expandable framework 12 to any degree (e.g., the full longitudinal extend of the expandable framework 12). In some cases, the occlusive covering 14 may be considered as including a front face 30 along a proximal extent of the occlusive member 14 and a periphery 32 that extends around the occlusive covering 14 at a position distal of the front face 30.

In some embodiments, the occlusive member 14 may be permeable or impermeable to blood and/or other fluids, such as water. In some embodiments, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. In some embodiments, the occlusive member 14 may prevent thrombi (i.e. blood clots, etc.) from passing through the occlusive member 14 and out of the LAA into the blood stream. In some embodiments, the occlusive member 14 may promote endothelialization after implantation, thereby effectively removing the left atrial appendage from the patient's circulatory system. Some suitable, but non-limiting, examples of materials for the occlusive member 14 are discussed below.

FIG. 1 further illustrates that the expandable framework 12 may include a plurality of anchor members 16 disposed about a periphery of the expandable framework 12. The plurality of anchor members 16 may extend radially outward from the expandable framework 12. In some embodiments, at least some of the plurality of anchor members 16 may each have and/or include a body portion and a tip portion projecting circumferentially therefrom. Some suitable, but non-limiting, examples of materials for the expandable framework 12 and/or the plurality of anchor members 16 are discussed below.

In some examples, the expandable framework 12 and the plurality of anchor members 16 may be integrally formed and/or cut from a unitary member. In some embodiments, the expandable framework 12 and the plurality of anchor members 16 may be integrally formed and/or cut from a unitary tubular member and subsequently formed and/or heat set to a desired shape in the expanded configuration. In some embodiments, the expandable framework 12 and the plurality of anchor members 16 may be integrally formed and/or cut from a unitary flat member, and then rolled or formed into a tubular structure and subsequently formed and/or heat set to the desired shape in the expanded configuration. Some exemplary means and/or methods of making and/or forming the expandable framework 12 include laser cutting, machining, punching, stamping, electro discharge machining (EDM), chemical dissolution, etc. Other means and/or methods are also contemplated.

As illustrated in FIG. 1, the plurality of anchor members 16 disposed along the expandable framework 12 may include two rows of anchor members 16. However, this is not intended to be limiting. Rather, the expandable framework 12 may include a single row of anchor members 16. In other examples, the expandable framework 12 may include more than two rows of anchor members 16. For example, in some instances the expandable framework 12 may include 1, 2, 3, 4 or more rows of anchor members 16. While FIG. 1 illustrates an expandable framework 12 which may be formed from a unitary member, this is not intended to be limiting. Rather, it is contemplated the expandable framework 12 may include a variety of different configurations which may be formed via a variety of manufacturing techniques.

As indicated above, the occlusive member 14 may include a woven fabric/material or mesh, a non-woven fabric/material or mesh, a braided and/or knitted material, a fiber, a sheet-like material, a fabric, a mesh, a fabric mesh, a polymeric membrane, a metallic or polymeric mesh, a porous filter-like material, a covering, and/or other suitable construction. The occlusive member 14 may be formed from a suitable material such as polyethylene terephthalate, polyester, nylon, acrylic materials, a polyolefin, and/or the like, combinations thereof, and/or other materials disclosed herein. In other instances, the occlusive material may include metallic mesh formed from nickel-titanium alloy, stainless steel, titanium, other materials disclosed herein, combinations thereof, and/or the like.

In some cases, a foam such as a shape memory polymer foam may be added to the LAAC device 10. In some cases, a shape memory polymer foam may be added to the occlusive member 14, for example. A shape memory polymer foam may be added to the face 30 of the occlusive member 14. A shape memory polymer foam may be added to the periphery 32 of the occlusive member 14. In some cases, the entire occlusive member 14 may be coated with a foamable solution. As noted above, a foamable solution may be added to the occlusive member 14 when the foamable solution is at Step One, i.e., a liquid pre-foam. A foamable solution may be added to the occlusive member 14 when the foamable solution is at Step Two, i.e., a partially reacted foam. A foamable solution may be added to the occlusive member 14 when the foamable solution is at Step Three, i.e., a fully reacted foam.

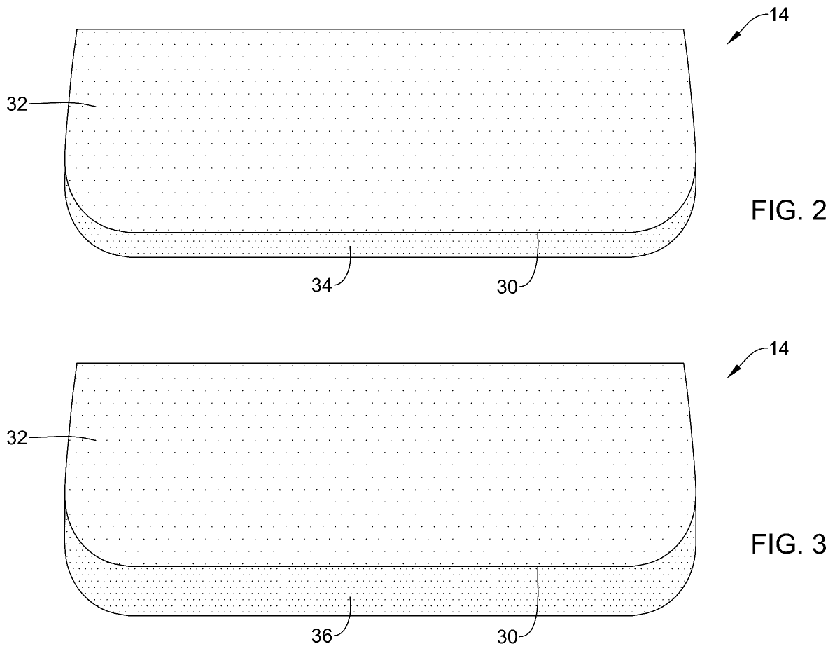

FIG. 2 is a schematic side view of the occlusive member 14, with the rest of the LAAC device 10 not shown for clarity. As shown, a foamable solution has been applied to the front face 30 of the occlusive member 14, forming a coating 34 on the front face 30. The foamable solution may be considered as having been applied at Step One. FIG. 3 shows the occlusive member 14 with the coating 34 having fully reacted into a shape memory polymer foam layer 36. While the shape memory polymer foam layer 36 has been shown schematically, it may be considered that since the occlusive member 14 may be porous, the shape memory polymer foam layer 36 may include fibers or strands of the occlusive member 14 that are encased within the shape memory polymer foam layer 36, thereby securing the shape memory polymer foam layer 36 to the front face 30 of the occlusive member 14.

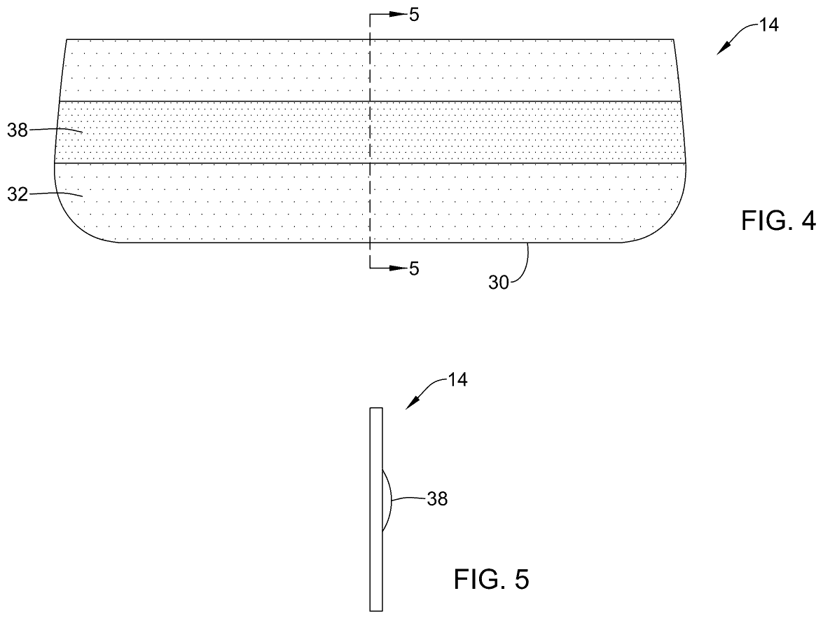

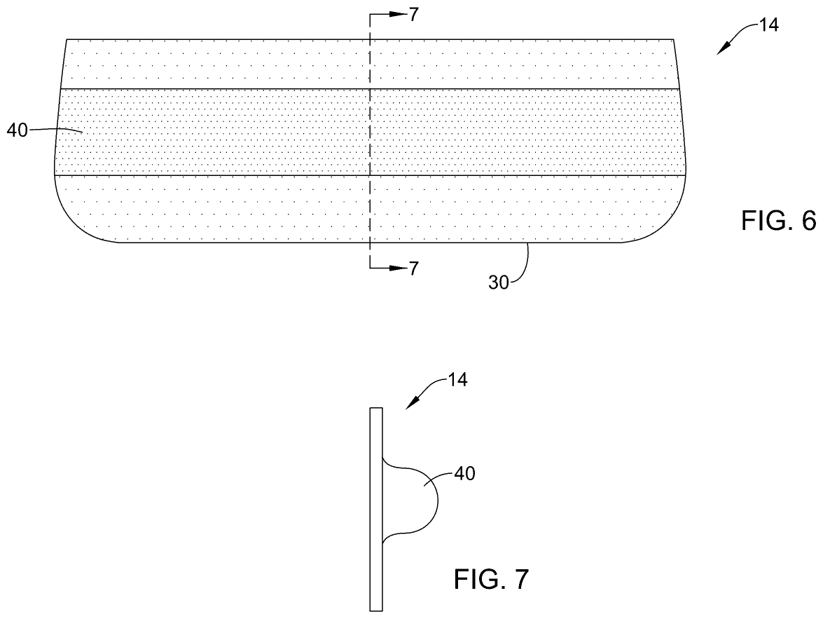

FIG. 4 is a schematic side view of the occlusive member 14, with the rest of the LAAC device 10 not shown for clarity, and FIG. 5 is a cross-sectional view taken along the line 5-5 of FIG. 4. As shown, a foamable solution has been applied to the periphery 32 of the occlusive member 14, forming a coating 38 on the periphery 32. The foamable solution may be considered as having been applied at Step Two. FIG. 6 shows the occlusive member 14 with the coating 38 having fully reacted into a shape memory polymer foam layer 40. FIG. 7 is a cross-sectional view taken along the line 7-7 of FIG. 6. While the shape memory polymer foam layer 40 has been shown schematically, it may be considered that since the occlusive member 14 may be porous, the shape memory polymer foam layer 40 may include fibers or strands of the occlusive member 14 that are encased within the shape memory polymer foam layer 40, thereby securing the shape memory polymer foam layer 36 to the periphery 32 of the occlusive member 14.

In some cases, a shape memory polymer foam layer may be formed over all of the occlusive member 14. In some cases, the shape memory polymer foam layer 36 may be formed on the front face 30 of the occlusive member 14 and the shape memory polymer foam layer 40 may be formed on the periphery 32 of the occlusive member 14. In some cases, the occlusive member 14 may be dipped into a prepolymer solution that includes multi-functional isocyanates and polyols to coat the occlusive member 14. The occlusive member 14 may then be contacted with an aqueous solution that includes one or more blowing agents and possibly one or more catalysts in order to complete the foaming reaction.

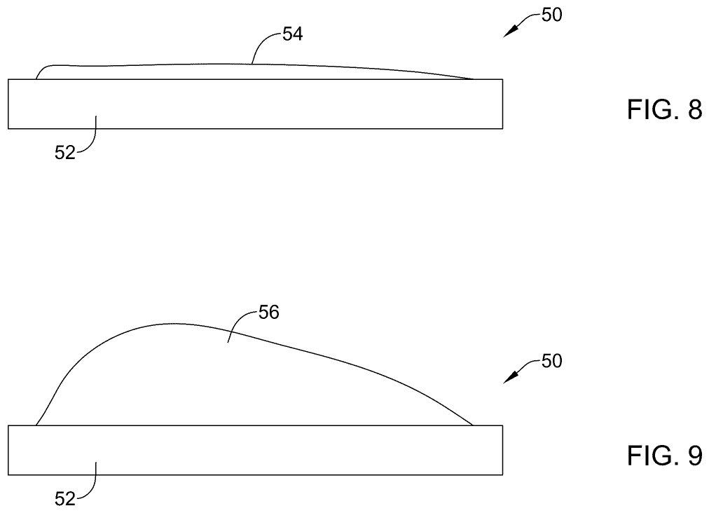

In some cases, a shape memory polymer foam layer may be added to any of a variety of medical devices. FIG. 8 is a schematic side view of an illustrative medical device 50 that includes a first component 52. The first component 52 may be a metallic component. The first component 52 may be a polymeric component. In some cases, the first component 52 may have undergone a surface treatment process. The first component 52 may be a non-shape memory polymer foam block. The first component 52 may be a shape memory polymer foam block. A foamable solution has been applied to the first component 52, to form a coating 54. In FIG. 9, the coating 54 has been fully reacted to form a shape memory polymer foam layer 56 on the first component 52. If the first component 52 is porous, the shape memory polymer foam layer 56 will adhere to the first component 52 by virtue of the coating 54 soaking partially into the first component 52 before the coating 54 is fully reacted. If the first component 52 is not porous, the shape memory polymer foam layer 56 may be adhered to the first component 52 by virtue of a surface treatment performed on the first component 52, or perhaps by using an adhesive between the first component 52 and the coating 54.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluorocthylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of making a medical device that includes a first component and a shape memory foam component secured relative to the first component, the method comprising:
forming a foamable solution that over time progresses from an initial liquid pre-foam state to a subsequent mid-foam state in which the foamable solution is partially reacted and to a final foam state in which the foamable solution is fully reacted; and
applying the foamable solution to the first component during one of the liquid pre-foam state, the subsequent mid-foam state and the final foam state.

2. The method of claim 1, further comprising allowing the foamable solution to progress to the final foam state.

3. The method of claim 1, wherein the pre-foam state corresponds to the foamable solution being less than about thirty percent reacted.

4. The method of claim 1, wherein the mid-foam state corresponds to the foamable solution being at least about thirty percent reacted and no more than about seventy percent reacted.

5. The method of claim 1, wherein the final foam state corresponds to the foamable solution being more than seventy percent reacted.

6. The method of claim 1, wherein:
the pre-foam state corresponds to the foamable solution being less than about twenty percent reacted;
the mid-foam state corresponds to the foamable solution being at least about twenty percent reacted and no more than about eighty percent reacted; and
the final foam state corresponds to the foamable solution being more than about eighty percent reacted.

7. The method of claim 1, further comprising applying a binding solution to the first component prior to applying the foam able solution to the first component.

8. The method of claim 7, wherein the binding solution comprises the same monomers as the foamable solution but without any blowing agents.

9. The method of claim 1, wherein the foamable solution comprises polyol or isocyanate monomers.

10. The method of claim 1, wherein the first component comprises an occlusive covering for a left atrial appendage closure (LAAC) device.

11. The method of claim 10, wherein applying the foamable solution comprises applying the foamable solution to a face of the occlusive covering during the foamable solution's initial pre-foam state.

12. The method of claim 10, wherein applying the foamable solution comprises applying the foamable solution to a periphery of the occlusive covering during the foamable solution's mid-foam state.

13. The method of claim 1, wherein the first component comprises a first block of foam.

14. The method of claim 1, wherein the first component comprises an anchoring system.

15. The method of claim 1, wherein the first component comprises a polymeric component and the method further comprises etching the polymeric component before applying the foamable solution.

16. The method of claim 1, wherein the foamable solution creates a shape memory foam.

17. A medical device produced by the method of claim 1.

18. A method of adding a shape memory polymer foam component to an implantable medical device, the method comprising:
forming a foamable solution;
allowing the foamable solution to reach one of an initial liquid pre-foam state, a subsequent mid-foam state and a final foam state; and
contacting the implantable medical device with the foamable solution once the foamable solution has reached the desired state.

19. The method of claim 18, wherein the implantable medical device comprises a left atrial appendage closure (LAAC) device.

20. A method of adding a shape memory foam component to a left atrial appendage closure (LAAC) device, the method comprising:
forming a foamable solution that over time progresses from an initial liquid pre-foam state to a subsequent mid-foam state in which the foamable solution is partially reacted and to a final foam state in which the foamable solution is fully reacted; and
applying the foamable solution to the LAAC device during one of the liquid pre-foam state, the subsequent mid-foam state and the final foam state.
